(19) United States
(12) Patent Application Publication
(10) Pub. No.: US 2012/0160896 A1
(43) Pub. Date: Jun. 28, 2012

(54) FASTENER FOR MECHANICAL SUTURE

(75) Inventor: William Houard, Labastide Rouairoux (FR)

(73) Assignee: TEXTILE HI-TEC (T.H.T.), Verreries De Moussans (FR)

(21) Appl. No.: 13/388,824

(22) PCT Filed: Aug. 5, 2010
(86) PCT No.: PCT/FR2010/051658

§ 371 Date: Mar. 16, 2012

(30) Foreign Application Priority Data
Aug. 6, 2009 (FR) 0955539

(51) Int. Cl. A61B 17/068; A61B 17/064

(52) U.S. Cl. 227/179.1; 606/219

(57) ABSTRACT

The fastener, in rest position, lies fully flat along a general plane. It is in a deformable material perpendicular to the said general plane so that it is able to be deformed at the time of implanting. Starting from its distal end, it comprises a curved part curved along at least a main arc of a circle and a terminal part extending the curved part and passing through or close to the centre of the main arc of a circle.

Advantageously, the curved part comprises:
a main part curved along the main arc of a circle of mean radius R1 over an angular portion A1 of at least 120°, and
an intermediate part, facing inwardly inside the main part, in an arc of a circle of mean radius R2 smaller than R1 over an angular portion A2.
   The applicator for the fastener comprises a storage and guide part comprising a helical housing in which the fastener is arranged in the deformed state.

### CROSS REFERENCE TO RELATED APPLICATIONS

This is a 371 national phase application of PCT/FR2010/051658 filed Aug. 5, 2010, claiming priority to French Patent Application No. 0955539 filed Aug. 6, 2009, the contents of which are incorporated herein by reference.

### FIELD OF THE INVENTION

The present invention concerns an implant for mechanical suture, also called a staple fastener, intended either to fix a prosthetic patch over tissues or to join together two groups of tissue for anastomosis.

### BACKGROUND OF THE INVENTION

The principle of mechanical suture is initially to achieve compression and immobilization of the tissues and subsequently the application and closure of the fasteners in the tissues. Numerous types of fasteners have already been proposed. Many are in the form of cylindrical coils.

In document FR 2 377 796 for example, the coiled fastener is used to join the edges of wound that have been drawn together over a certain length, the coils of the staple enter alternately into either side of the edge of the wound. The placing in position of said fastener is achieved using a hollow needle having the same cylindrical coil configuration, inside which the fastener is contained. For placing in position, the hollow needle is moved in rotation to draw the edges of the wound together then the needle is retracted to leave in place the fastener previously contained therein.

In document EP 0 121 362, the fastener is in the form of a loop having two free ends arranged laterally with respect to each other; this loop may be formed of a single coil or several coils. When it is placed in position, the fastener is in the form of a straight section, being arranged longitudinally inside a hollow needle whose distal part is curved. It is the forced passing of the straight section over this distal curved portion which imparts a loop configuration to the fastener as can be clearly seen in FIG. 6 of this document EP 0 121 362. The placing in position of the fastener requires the practitioner to position the distal curved portion transversally relative to the line formed by the joined edges of the wound. Once in place, the fastener lies substantially in a plane transverse to this line.

Another type of fastener is known from document EP 0 773 742 to ligate tissues or to attach an implantable device. This fastener comprises a continuous helical coil whose distal end has a tip able to pierce tissues. This fastener, at its proximal end, comprises a T-shaped bar intended to engage with an applicator for insertion of this fastener into the tissues. The placing in position of the fastener is performed in a direction substantially perpendicular to the plane of the tissues in which it is to be implanted. The number of helical coils and the pitch thereof are dependent on the cases to be treated. In this document, the fastener when at rest has a helical coil three-dimensional configuration.

The present invention lies in the technical field of the type of fastener taught in document EP 0 773 742. It is considered that the fastener described in this document has at least two shortcomings, one related to the necessary thickness to obtain good securing, a thickness which generally requires several consecutive coils. Said thickness may generate pain or discomfort for the patient. Another shortcoming is related to a practical viewpoint, the use of special tools to obtain the forming of this staple, in particular its three-dimensional forming.

### SUMMARY OF THE INVENTION

The objective of the present invention is to overcome the above-mentioned shortcomings. This objective is fully achieved with the fastener for mechanical suture which in manner known per se has a distal end able to penetrate the tissue and to pierce a prosthetic patch. Characteristically according to the present invention, this fastener in rest position lies fully flat over a general plane; in addition, it is in elastically deformable material at least in the direction perpendicular to the said general plane so that it is able to undergo such deformation when being implanted. Finally it comprises, from its distal end, a part that is curved at least over a main arc of a circle and a terminal part extending the curved part and passing through or close to the centre of the main arc of a circle.

Therefore in accordance with the particular arrangements given above, the fastener in rest position does not have a three-dimensional configuration as in the aforementioned documents, including document EP 0 773 742, which means firstly that the manufacture thereof is simplified, in particular it can be obtained by mere cut-out e.g. by water jet cutting from a planar sheet, and secondly it can be stored under optimal conditions. In addition, at the time of its implanting, the fastener of the present invention is inserted into the tissues whilst undergoing deformation perpendicular to its general plane when at rest. Implantation is performed starting from the distal end, subjecting the fastener to rotation about itself until its terminal part comes to bear against the outer side of the tissues or preferably the outer side of the prosthetic patch which is to be fastened to the tissues by means of the said fastener. Once the fastener is implanted, since the constituent material of the fastener is elastically deformable, the said material tends to resume its rest position so that, if the fastener is used to fasten a prosthetic patch, this patch is held in place by clamping under the elastic action of the fastener which is able to anchor itself more or less over a horizontal plane. This makes it possible to use the fastener on tissues of very small thickness, for example on the vagina.

According to one variant of embodiment, the curved part successively comprises from the distal end:
a main part curved in the main arc of a circle, of mean radius R1 on an angular portion A1 of at least 120°, and
an intermediate part in an arc of a circle, of mean radius R2 smaller than R1 on an angular portion A2, the said intermediate part facing inwardly inside the main part.

The value of 120° at least for the angular portion A1 is necessary so that, at the time of deformation of the fastener for insertion thereof, there is sufficient clearance height-wise for good tissue penetration.

Preferably, the angular portion A1 is between 120° and 359°. The value of 359° for this angular portion A1 corresponds to a particular embodiment in which this curved part is almost fully curved over itself, the end part extending substantially radially.

In one variant of embodiment, with an intermediate part in an arc of a circle of mean radius R2, the angular portion A2 is equal to or less than 290°.

In one variant of embodiment, the mean radius R1 is substantially two to three times the mean radius R2.

In one variant of embodiment, the end portion is rectilinear and passes through the centre of the arc of a circle of the main part.

In one variant of embodiment, the curved part, particularly the intermediate part, passes close to the distal end, the angular portion A1 particularly being at least 300°. This variant provides for maximum deformation of the fastener, in the direction perpendicular to its general plane when at rest.

Preferably, the distal end is bevelled, with an oblique planar face which, when the fastener is implanted, faces backwards. Preferably this oblique face cuts through the general plane of the fastener when at rest along a straight line which passes through or close to the centre of the main arc of a circle.

The fastener of the present invention can be obtained by bending a wire of substantially circular cross-section. It can also be obtained, since it is fully flat when at rest, by cutting a planar sheet in particular using water jet.

It is another objective of the present invention to propose an applicator specially designed for implanting the above-mentioned fastener. This device comprises:
a tubular body,
a part for storing and guiding at least one fastener, comprising a helical-shaped housing in which the fastener is arranged in the deformed state along an angular portion A, said part being fixed onto the inner periphery of the tubular body,
a driving part arranged longitudinally inside the storage and guide part and comprising a longitudinal notch for placing and driving the end part of the fastener,
means for diving the driving part in rotation about itself, independently of the tubular body, at an angle A,
a fastener discharge head closing the tubular body and comprising a first tangential opening for ejecting the distal end and at least the main part of the fastener, and a second front opening in the continuation of the first tangential opening for ejecting at least the terminal part of the fastener.

In addition, characteristically, the second front opening is offset height-wise relative to the base of the first tangential opening of the discharge head, and the discharge head has outer guiding faces for the fastener when it is implanted, said faces having a helical slope and extending from the base and the inner side of the first tangential opening substantially as far as the second front opening.

Therefore the fastener, which when at rest is of planar shape, is deformed for positioning thereof in the helical-shaped housing of the storage and guide part, this deformation occurring in the direction perpendicular to the general plane of the fastener at rest. In addition, it is necessary that at the fastener outlet, this deformation should be maintained until completion of the insertion of the fastener in the tissues and optionally the prosthetic patch i.e. until the release of the end part of the fastener. This is obtained by means of the presence of the outer, helical-shaped guiding faces of the discharge head which extend from the base and the inner side of the first tangential opening, via which the distal end and at least the main curved part of the fastener emerge, as far as substantially the second front opening via which the end part of the fastener emerges.

According to one variant of embodiment, the driving part comprises a central axial stud which extends, inside the discharge head, beyond the first tangential opening as far as the second front opening. This axial stud acts as a form of bearing for rotation of the driving part at the discharge head.

The applicator device comprises a shell having an opening through which the driving part passes. Additionally, the driving means comprise:
an actuating handle, pivoting at an angle about a fixed pin at an angle B,
transforming means capable of transforming the said angular pivoting at an angle B into rotation of the driving part about itself at an angle A, and,
a backstop system preventing the reverse rotation of the driving part when the handle returns to its original position.

In one preferred variant of the embodiment, the transforming means comprise:
a freely rotating toothed wheel,
a first curved rack on the edge of the actuating handle,
a freely rotating twisted bar, secured to the driving part via the backstop system,
a transforming cage through which the twisted bar passes, comprising two inner side casters which come into contact with the twisted bar either side thereof, and a second rack arranged longitudinally relative to the twisted bar on the outer face of the said cage.

The different elements mentioned above are arranged so that the first rack, when the handle pivots at an angle B, drives the rotation of the toothed wheel, this wheel driving the longitudinal travel of the second rack and hence of the transforming cage, said movement itself causing the setting in rotation of the twisted bar and hence of the driving part at an angle A.

According to one preferred variant of embodiment, the backstop system comprises:
a first ring fixed around a distal, non-twisted portion of the twisted bar,
a second ring mounted slidingly on said distal portion, held away from the first ring by a spring and comprising a distal friction face,
a friction plate mounted on the free proximal end of the driving part.

All these elements are arranged so that the setting in rotation of the driving part only takes place when the friction plate is in contact with the distal friction face, in particular due to applying of the discharge head onto the point at which the implant is to be implanted. Therefore, when the practitioner wishes to implant a fastener, the discharge head of the applicator device must necessarily be applied with some amount of pressure allowing compression of the spring which extends between the first and second rings, to cause the distal friction face to come into contact with the friction plate, which triggers rotation of the driving part and hence the release of the fastener. Once the fastener is inserted, the practitioner releases this pressure, the effect of which is to separate the twisted bar and driving part preventing the ejection of a new fastener when the actuating handle returns to its initial position, the twisted bar rotating in reverse direction but without driving the driving part with this rotation.

The risk subsists, however, that further to a user error the practitioner may maintain the application of this pressure whilst releasing the actuation handle. In this case, the release of the handle would cause the rotation in opposite direction of the twisted bar and also of the driving part. To prevent this risk, the applicator device of the present invention may additionally comprise a blocking finger and the second ring may comprise longitudinal grooves forming safety catches, the said grooves being configured to allow the passing of the blocking finger from one groove to another when the actuating part is rotated by an angle A to eject a fastener, and to act as abutment for the blocking finger when the actuating part is rotated in the opposite direction.

It is another objective of the present invention to propose a fastener charger assembly formed of the tubular body, the storage part, the actuating part and the discharge head, as mentioned above, the said charger assembly being independent of the shell and able to be joined therewith by press-fitting the actuating part in the backstop system, in particular by press-fitting the friction plate in the second ring.

### BRIEF DESCRIPTION OF THE DRAWINGS

The present invention will be better understood on reading the following description of several examples of fasteners for technical suture having the aforementioned structural characteristics and of a preferred example of embodiment of the applicator device allowing the implanting of this fastener in the deformed state, in a direction perpendicular to the general plane of the fastener when it lies flat, with reference to the appended drawings in which:

FIGS. 1 to 3 are schematic views of a first example of a fastener, respectively a front view of the fastener at rest in FIG. 1, a side view at rest in FIG. 2 and a side view in the deformed state for storing and implanting in FIG. 3;

FIGS. 4 to 13 are schematic front views of ten other examples of fasteners;

FIG. 14 is a schematic side view of a charger assembly;

FIG. 15 is a partial view of the charger assembly in FIG. 14, exposing the inside of the said assembly;

FIG. 16 is a perspective view of the storage and guide part;

FIG. 17 is a perspective view of the driving part;

FIG. 18 is a perspective view of the discharge head;

FIGS. 19 to 24 are partial, perspective views of the charger assembly close to the discharge head, giving a breakdown of the different positions of the fastener as and when it is implanted,

FIG. 25 is a schematic perspective view of the applicator gun, with the shell open,

FIGS. 26 and 27 are schematic perspective views of the transforming means, giving an underside view in FIG. 26, and a rear view in FIG. 27.

### DETAILED DESCRIPTION

The fastener for mechanical suture of the present invention has the first particular aspect that when at rest it does not have a three-dimensional configuration, as is the case for the other fasteners described in documents FR 2 377 796, EP 0 121 362 and EP 0 773 742, but it is fully flat able to rest on a plane which, herein, is considered to be the general plane of the fastener when flat or at rest.

This fastener is in a material which is elastically deformable a least in a direction perpendicular to the said general plane of the fastener when flat. It is under said deformation that the fastener is maintained throughout the entire implanting operation in the tissues.

In the first example of embodiment, the fastener 1 comprises a distal end 2 which is conformed so that it is able to penetrate the tissues easily and also to pierce a prosthetic patch.

Starting from this distal end 2, the fastener comprises a curved part 3 curved in at least a main arc of a circle 5, and a terminal part 4 which extends the curved part 3 and which passes through the centre 6 of the main arc of a circle 5 or close thereto. In this first example the curved part 3 successively comprises, from the distal end 2, firstly a main curved part 7 on the main arc of a circle 5 having a mean radius R1 over an angular portion A1 of at least 120°, and then an intermediate part 8 in an arc of a circle of mean radius R2, smaller than R1, over an angular portion A2. The intermediate part 8 faces inwardly inside the main part 7 so that the terminal part 4 is effectively able to pass through the centre 6 of the main arc of a circle 5 or at least close thereto.

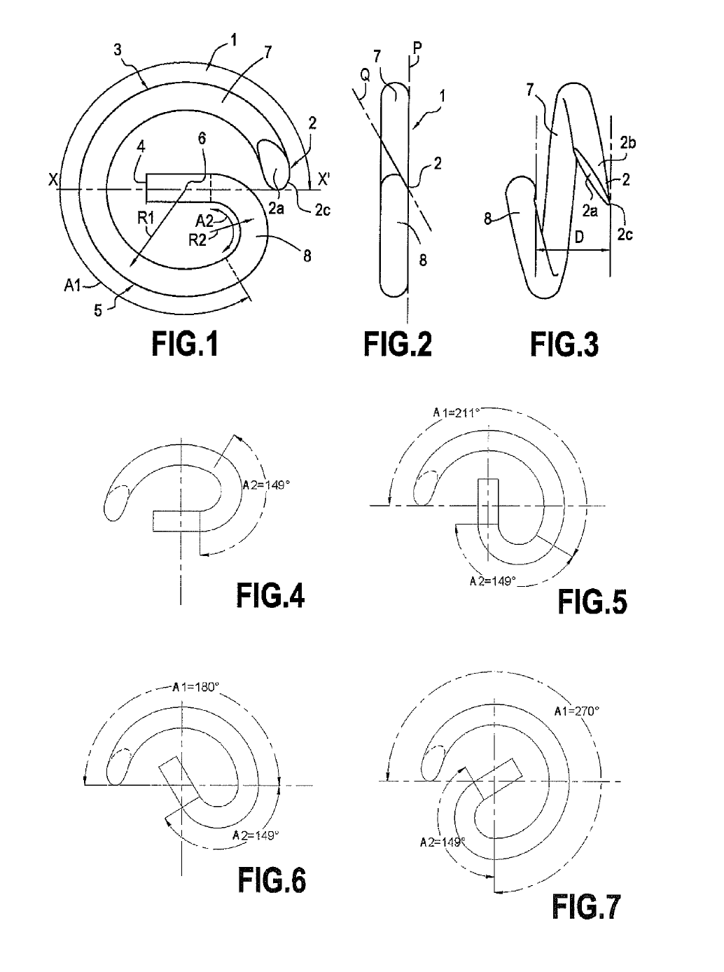

In this first example illustrated in FIG. 1, the main part 7 occupies an angular portion A1 of 301° and a mean radius R1 of 1.2 cm, whilst the intermediate part 8 occupies an angular portion A2 of 149° and has a mean radius R2 of 0.55 cm. It is to be considered that the maximum volume taken up by the fastener, when it is laid flat along its general plane at rest, corresponds to the space which would be taken up by the part curved along the main arc of a circle over a complete angular portion of 360°.

The presence of the intermediate part 8, curved along an arc of a circle whose mean radius R2 is smaller than the radius R1 of the main part 7, allows the deformation needed for implanting under good conditions to be achieved without any exaggerated risk of fastener rupture at the join between the terminal part 4 and the main curved part 7.

FIG. 2 shows the fastener 1 illustrated in FIG. 1 at rest. It is perfectly flat, the curved part 3 and the terminal part 4 able to be in contact with the general plane P over the entire length of the fastener.

FIG. 3 shows the configuration of the fastener 1 in its deformed state, such as is necessarily assumed for its implantation, a deformation which leads to distancing, by a distance D, the distal end 2 from the terminal part 4.

The fastener can be formed, as illustrated in FIGS. 1 to 3, from a wire of substantially circular cross-section, by bending. In this case, the distal end 2 can be formed by a bevel cut to leave an oblique planar face 2a and a curved face 2b which, for this distal end 2, delimit a curved tip 2c capable of penetrating tissues and piercing an optional prosthetic patch.

The plane Q of the oblique face 2a of the distal end 2 of the fastener at rest cuts through the general plane P of the fastener at rest along a straight line XX' which passes through the centre 6 of the main arc of a circle 5 formed by the main curved part 7, or close to the said centre 6.

As can be clearly seen in FIG. 3, at the time of implanting the fastener 1, the curved face 2b is oriented forwards i.e. in the direction of implanting, whilst the oblique planar face 2a is oriented backwards. This has the effect of orienting the implanting of the fastener as it penetrates the tissues, the oblique face 2a tending to drive the fastener deep into the tissues.

This first example of a fastener 1 is given as a non-limiting example, even if it is a preferred embodiment. FIGS. 4 to 13 show ten other examples of embodiment which chiefly differ through the value of the angular portions Al and A2 and the value of the mean radii R1 and R2, more specifically through the R1/R2 ratio of the said radii, since the absolute value of the mean radii itself depends on the desired size of the fastener. In Table 1 below each of the values A1, A2, and R1/R2 are given for the ten examples under consideration, referenced Ex 2 to Ex 11 for each of FIGS. 4 to 13 respectively, and for the first example Ex 1 in FIG. 1. It is also specified in the Table whether the fastener has a rectilinear (R) or curved (C) terminal part.

TABLE 1

| Ex | FIG | A₁ | A₂ | R₁/R₂ | (R) or (C) |
|---|---|---|---|---|---|
| Ex 1 | 1 | 301° | 149° | 2.18 | (R) |
| Ex 2 | 4 | 121° | 149° | 2.11 | (R) |
| Ex 3 | 5 | 180° | 149° | 2.50 | (R) |
| Ex 4 | 6 | 211° | 149° | 2.14 | (R) |
| Ex 5 | 7 | 270° | 149° | 2.40 | (R) |
| Ex 6 | 8 | 359° | — | — | (R) |
| Ex 7 | 9 | 333° | 117° | 5.6 | (R) |
| Ex 8 | 10 | 322° | 270° | 2.07 | (C) |
| Ex 9 | 11 | 301° | 264° | 1.53 | (C) |
| Ex 10 | 12 | 270° | 262° | 1.33 | (C) |
| Ex 11 | 13 | 270° | 262° | 1.39 | (R) |

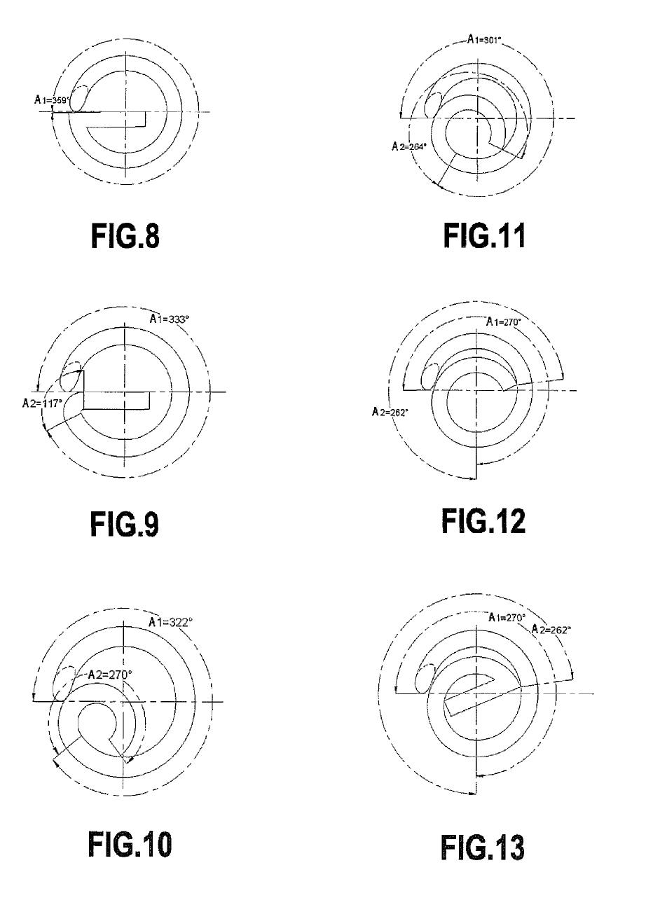

Manufacturing by mere bending can be envisaged for examples Ex 6 and Ex 11, in which the terminal parts are joined in the first example to the main curved part (Ex 6, FIG. 8) and in the second to the intermediate part (Ex 11, FIG. 13) via a connecting region which is not curved. In this case, and in others in which it may prove to be complex, manufacturing uses the cutting of plate in particular by water jet. This manufacturing technique is possible in all configurations since the fastener at rest is always flat, only being deformed when it is implanted and optionally during storage.

An applicator has been specially designed to allow the implanting of the fastener 1 of the invention. This applicator comprises a device of gun type on which a charger assembly can be adapted by mere press-fitting which contains a plurality of fasteners 1 in their deformed state.

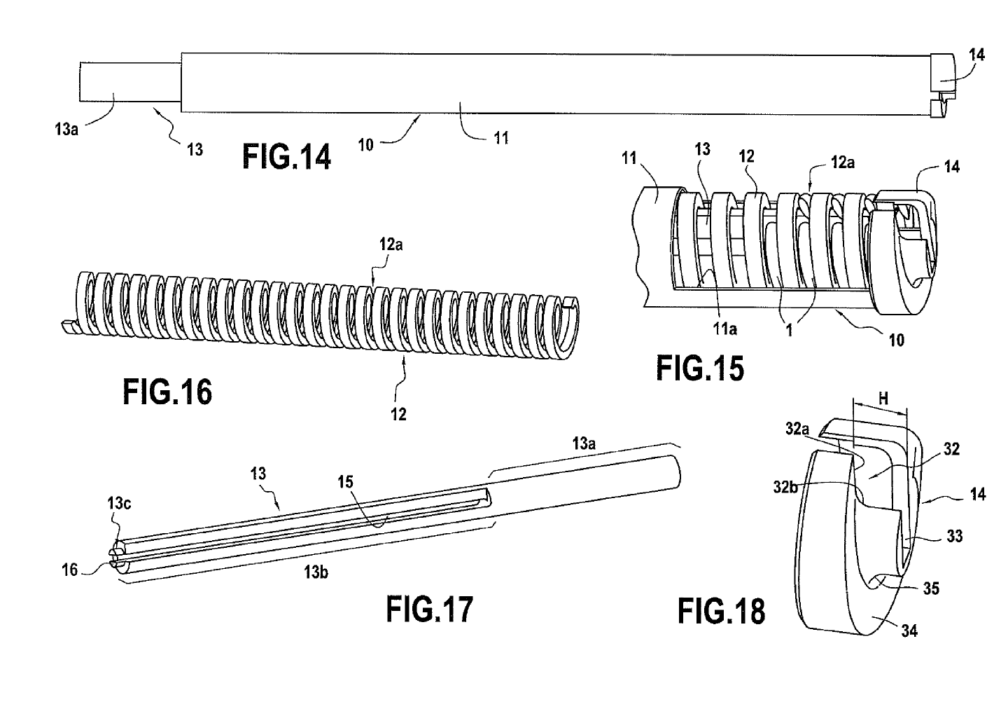

The charger assembly 10 (FIG. 14) is composed of four parts, namely a tubular body 11, a storage and guide part 12 (FIG. 16), a driving part 13 (FIG. 17) and a discharge head 14 (FIG. 18).

The storage and guide part 12 comprises a housing 12a which is continuous for all the fasteners following after one another (FIG. 15) and which is helical-shaped so that each fastener 1 is necessarily deformed when it lies inside the said housing 12a, This storage and guide part is fixed on the inner periphery 11a of the tubular body 11, preferably without any particular securing means but by mere friction between the components of these two elements.

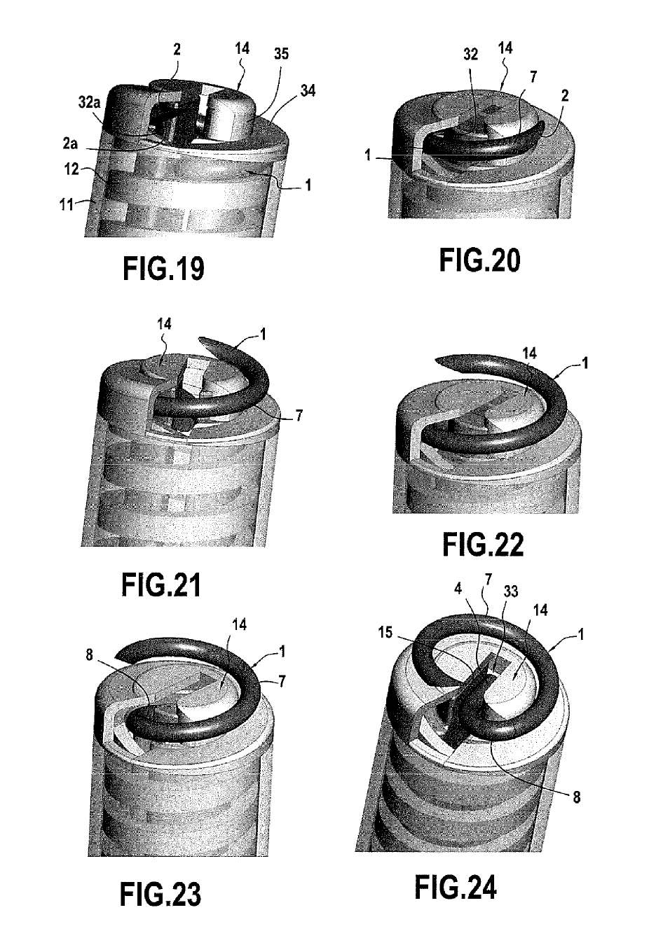

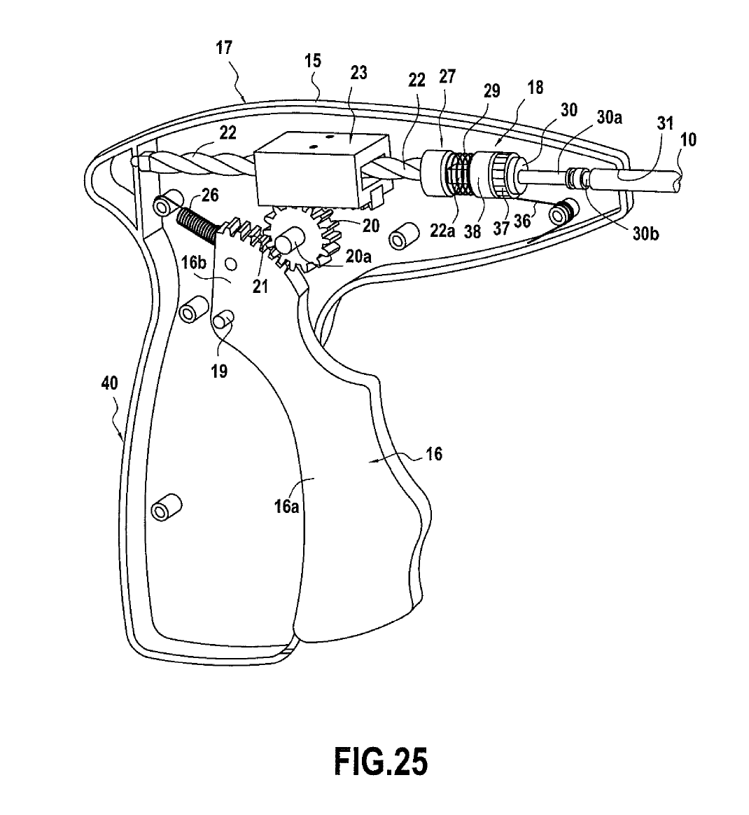

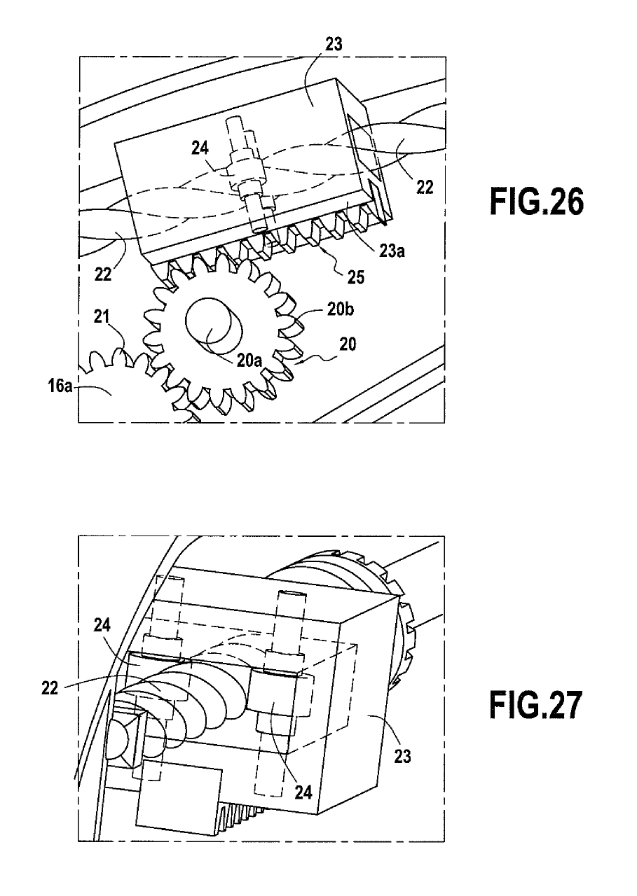

The driving part 13 is a solid single-piece part, globally cylindrical, having a proximal part 13a, an intermediate part 13b and a distal part 13c, In the charger assembly 10, this driving part is mounted longitudinally inside the storage and guide part 12. Its cylindrical proximal part 13a partly projects beyond the tubular body; it is this proximal part 13a which is used to adapt the charger assembly 10 onto the gun. Its intermediate part 13b comprises a longitudinal notch 15 which is used for placing and driving the terminal part 4 of the fastener 1. Its distal part 13c, of short length, is also cylindrical but of smaller diameter; it is in the form of a stud 16 acting as bearing for rotation of the driving part 13 inside the discharge head 14. The discharge head 14 is press-fitted onto the front end of the tubular body 11. To allow the passing of the fastener, it has two openings each in the continuation of the other: a first tangential opening 32 and a second front opening 33, which is offset by a height H relative to the base 32a of the first tangential opening 32. The discharge head 14 has two helical outer faces 34, 35, which extend respectively from the base 32a and from the inner side 32b of the front opening 32 substantially as far as the second front opening 33, in the continuation of the helical housing 12a of the storage and guide part 12. It is these outer faces 34, 35 which are used to guide the fastener maintained in its deformed state throughout the entire duration of its release and implantation, as is described below with reference to FIGS. 19 to 24.

To cause the release of the fastener 1 the closest to the discharge head 14 and hence the implanting thereof, the proximal part 13a of the driving part 13 of the charger assembly 10 must be driven in rotation by a determined angle A, of 360°. This setting in rotation is performed by means of the gun 40 which comprises a shell 15, an actuating handle 16 which partly projects beyond the shell 15, and inside the shell there are inner transforming means 17 and a backstop system 18 on which the charger assembly 10 is connected via the said proximal part 13a.

The actuating handle 16 is pivot-mounted relative to a fixed pin 19. It comprises, relative to the pin 19, a lower part 16a, which projects partly beyond the shell 15 and which is actuated by the practitioner, and an upper part 16b, shorter than the lower part 16a, secured to the shell 15 via a return spring 26.

The function of the inner transforming means 17 is to transform the angular movement by angle B of the handle 16 to a movement in rotation allowing the driving part 13 to be caused to rotate about itself by an angle A. These means are formed of:
a toothed wheel 20, mounted in free rotation about a pin 20a parallel to the pin 19 of the handle 16,
a first curved rack 21 concentric relative to the pin 19, which is arranged on the edge of the upper part 16b of the handle 16,
a twisted bar 22 whose cross-section comprises a least one bearing face, this twisted bar being mounted in free rotation by means of bearings arranged on the inner faces of the shell and being secured to the proximal part 13a of the driving part 13 via the backstop system 18,
and finally a transforming cage 23.

The said cage 23 globally has the configuration of a rectangular parallelepiped. The twisted bar 22 passes through it and it comprises at least one preferably two inner casters 24 which laterally come into contact with the preferably two bearing faces of the twisted bar either side thereof. The lower face 23a of the cage 23 is equipped with a second rack 25. The serrations of the first rack 21 of the handle 16 and of the second rack 25 of the cage 23 engage with the teeth 20b of the toothed wheel 20. When at rest, the lower part 16a of the handle 16 partly projects beyond the shell 15, under the effect of the return spring 26. When the practitioner applies maximum pressure to the handle 16, the upper part 16b is caused to rotate and hence the first rack 21 by an angle B about the pin 19. The movement of the first rack 21 turns the toothed wheel 20 which causes the longitudinal movement of the second rack 25 and hence of the cage 23. During this movement, the two casters 24 which are in close contact on the opposite bearing faces of the twisted bar 22 force the latter to pivot about itself.

The backstop system 18 ensures the rotational connection between the twisted bar 22 and the charger assembly 10, when the rotation of the twisted bar is due to actuation of the handle 16 by the practitioner, for example in clockwise direction. On the other hand it annihilates this connection when the rotation of the twisted bar in opposite direction is due to release of the handle and its return to rest position under the action of the return spring 26. This backstop system 18 is composed of a first ring 27 fixed around a distal non-twisted portion 22a of the twisted bar 22, a second ring 28 mounted slidingly in this non-twisted distal portion and comprising a distal friction face, a spring 29 arranged between the two said rings 27, 28 which holds the second ring 28 away from the first ring 27 in rest position, and finally a friction plate 30 secured to the driving part 13 of the charger assembly 10. So that rotation of the driving part 13 may take place, the second ring 28 must necessarily be caused to slide along the distal portion of the twisted bar until the spring 29 is compressed and the friction plate 30 is in frictional contact with the friction face of the second ring 28.

In the present detailed example, the friction plate 30 is extended towards the front opening 31 of the shell via a longitudinal rod 30a which, at its end opposite the friction plate 30, comprises a hollow cylindrical end-piece 30b conformed to receive by press-fitting the proximal part 13a of the driving part 13.

The implementing of the ready-to-use applicator device by the practitioner for implanting one or more fasteners 1 is as follows. The practitioner takes hold of the gun 40 previously fitted with a charger assembly 10, the proximal part 13a of the driving part 13 of the charger assembly having been previously inserted in the front opening 31 of the gun 40 until the said proximal part 13a press fits into the cylindrical end-piece 30b, The gun 40 is directed so as to place the discharge head 14 of the charger assembly 10 onto the point where the fastener is to be implanted. The gun 40 is applied with a certain amount of pressure to obtain compression of the spring 29 and frictional contact between the friction plate 30 and the friction face of the second ring 28. By actuating the handle 16 as far as possible, as described previously, the practitioner obtains the setting in rotation about itself of the friction plate 30 and hence of the cylindrical end-piece 30b and of the driving part 13 at an angle A which is determined to obtain release of a fastener via the discharge head 14 of the charger assembly 10, this release occurring when the fastener 1 is in its deformed state as illustrated in FIG. 3 and as it is driven forwardly and in rotation.

FIGS. 19 to 24 give the breakdown of the movement of the fastener 1 caused by the rotation by angle A of the driving part 13 inside the charger assembly 10. It is to be noted that the tubular body 11 and the storage and guide part 12, secured to the tubular body, are held in a fixed position on account of the pressure applied to the discharge head 14 which prevents them from entering into rotation. The distal end 2 of the fastener 1 starts by leaving via the first tangential opening 32, the oblique bevelled face 2a of this distal end being oriented towards the base 32a of the said first tangential opening 32 (FIG. 19). The first curved part 7 gradually leaves via the first opening 32 following a helical movement on the outer guide faces 34, 35 (FIGS. 20, 21, 22). The curved intermediate part 8 of the fastener then moves out of the first opening 32 (FIG. 23). Finally, since the longitudinal notch 15 of the driving part 13 lies opposite the second opening 33, the terminal part 4 of the fastener emerges via the said second front opening 33.

The penetration of the fastener 1 through the prosthetic patch and then the tissues occurs during the phases illustrated in FIGS. 20, 21 and 22. The depth of this penetration is dependent upon the pressure exerted by the practitioner and on the deformation capability of the tissues. The anchoring of the prosthetic patch takes place when the terminal part 4 is released which, on account of its configuration, cannot penetrate either the patch or the tissues but it ensures the clamping of the prosthetic patch in the tissues. Penetration can occur over a reduced tissue thickness since it takes place at the time of attack by the distal end 2 at an angle which may a grazing angle. It is possible to obtain penetration along a plane that is practically horizontal in the tissues. In addition, since the fastener is in the deformed state during this penetration and since it is in an elastically deformable material, after it has been released it tends to return gradually to its initial flat configuration, which allows a clamping effect to be obtained between whatever lies between the distal end 2 and the terminal part 4, and therefore between the prosthetic patch and the tissues.

The fastener of the present invention can be formed from a non-absorbable material whether or not metallic. Preferably it is in an absorbable material whose mechanical properties in terms of rupture strength change over time so that six months after implanting there is a loss of 50% or less of its initial properties, and after one year this loss is total. It may in particular be a polymer of natural or synthetic origin, such as polylactide, polyglycolide, polys-caprolactone, polyhydroxybutyrate, polyhydroxyvalerate, polycarbonate and the like, cellulose, polysaccharide, starch or a homopolymer or copolymer or a derivative of the above-mentioned polymers, whether or not containing a filler. If a filler is used, it is preferably hydroxyapatite/PTCP in powder and/or nanopowder form to the proportion of 5 to 60 weight %.

When the gun 40 is used by the practitioner, a difficulty may arise if the practitioner releases the actuating handle 16 whilst maintaining certain pressure on point of implantation, holding the spring 29 compressed and hence the friction plate and friction face in more or less frictional contact. In this case, the return of the handle 16 towards its rest position under the action of the return spring 22 may cause some rotation of the driving part in opposite direction, thereby moving backwards the fasteners contained in the charger assembly. Since this reverse rotation may take place at an angle different from angle A, there may be an offset preventing release of the fasteners under the optimal conditions described above. To overcome this difficulty, the backstop system 18 comprises blocking means preventing this rotation in reverse direction, even if pressure is maintained. These means, in the example of embodiment previously described, comprise a blocking finger 36 and longitudinal grooves 37 formed in the second ring 28 and in which the said blocking finger 36 is able to enter during rotation of the twisted bar 22 and of the driving part 13. These longitudinal grooves 37 are configured in semi-V shape so as to allow the passing of the blocking finger 36 from one groove 37 to another when rotation occurs in the proper direction for release of the fasteners by merely passing over the inclined face of the semi-V, and so as to form an abutment for the blocking finger 36 if rotation occurs in the opposite direction by coming to bear upon the vertical face of the semi-V.

1. A fastener mechanical suture, having a distal end capable of penetrating tissues and of piercing a prosthetic patch, the said fastener in rest position being fully flat along a general plane, and comprising from its distal end, a curved part curved in at least a main arc of a circle, wherein it is in a mechanically, elastically deformable material at least in the direction perpendicular to the said general plane so that it is able to undergo such deformation at the time of implanting and so that it tends gradually to return to its rest position after implantation, and wherein it further comprises a terminal part extending the curved part and passing through or close to the centre of the main arc of a circle.

2. The fastener according to claim 1, wherein the curved part successively comprises, from the distal end:
a main part curved along the main arc of a circle, of mean radius R1 over an angular portion A1 of at least 120°, and
an intermediate part in an arc of a circle of mean radius R2 smaller than R1 over an angular portion A2, said intermediate part facing inwardly inside the main part.

3. The fastener according to claim 1, wherein the angular portion A1 is between 120° and 359°.

4. The fastener according to claim 2, wherein the angular portion A2 is equal to or less than 280°.

5. The fastener according to claim 1, wherein the mean radius R1 is substantially two to three times the mean radius R2.

6. The fastener according to claim 1, wherein the terminal portion is rectilinear and passes through the centre of the arc of a circle of the main part.

7. The fastener according to claim 1, wherein the curved part in particular the intermediate part passes close to the distal end, the angular portion A1 being in particular at least 300°.

8. The fastener according to claim 1, wherein the distal end is bevelled, with an oblique face which cuts through the general plane of the fastener at rest along a straight line passing through or close to the centre of the main arc of a circle.

9. The fastener according to claim 1, wherein it is obtained by bending a wire of substantially circular cross-section.

10. The fastener according to claim 1, wherein it is obtained by cut-out from a plate, in particular using water jet.

11. An applicator device specially designed for implanting the fastener according to claim 1, where it comprises:
a tubular body,
a storage and guide part for at least one fastener, comprising a helical-shaped housing in which the at least one fastener is arranged in the deformed state over an angular portion A, said part being fixed on the inner periphery of the tubular body,
a driving part arranged longitudinally inside the storage and guide part and comprising a longitudinal notch for placing and driving the terminal part of the fastener,
means for driving the driving part in rotation about itself, independently of the tubular body, by an angle equal to A,
a discharge head for the fastener, closing the tubular body and comprising a first tangential opening for discharging the distal end and at least the main part of the implant, and a second front opening, in the continuation of the first tangential opening for discharging at least the terminal part of the fastener.

12. The device according to claim 11, wherein the discharge head is in the form of a cover which press-fits onto the open distal end of the tubular body.

13. The device according to claim 11, wherein the second front opening is offset height-wise relative to the base of the first tangential opening of the discharge head and wherein the said discharge head has outer guide surfaces for the fastener at the time of its implantation, the said helical-shaped faces extending from the base and the inner side of the first tangential opening substantially as far as the second front opening.

14. The device according to claim 11, wherein the driving part comprises a central axial stud axial extending inside the discharge head beyond the first tangential opening as far as the second front opening.

15. The device according to claim 11, wherein the storage and guide part is fixed to the inner periphery of the tubular body by mere friction, without any other mechanical securing means.

16. The device according to claim 11, wherein it comprises a shell having an opening through which the driving part passes, and wherein the driving means comprise:
an actuating handle with angular pivoting about a fixed pin at an angle B,
transforming means capable of transforming the said angular pivoting at angle B to rotation of the driving part about itself at an angle A, and
a backstop system preventing the reverse rotation of the driving part when the handle returns to its original position.

17. The device according to claim 16, wherein the transforming means comprise:
a freely rotating toothed wheel,
a first curved rack on the upper edge of the actuating handle,
a freely rotating twisted bar secured to the driving part via the backstop system,
and a transforming cage through which the twisted bar passes, comprising two inner lateral casters coming into opposite-facing contact with the twisted bar, and a second rack arranged longitudinally relative to the twisted bar on the underside of the said cage,
so that the first rack, when the handle pivots at an angle B, causes the rotation of the toothed wheel, which causes the longitudinal movement of the second rack and hence of the transforming cage, which causes the setting in rotation of the twisted bar and hence of the driving part at an angle A.

18. The device according to claim 17, wherein the backstop system comprises:
a first ring fixed around a distal non-twisted portion of the twisted bar,
a second ring slidingly mounted on said distal portion, held away from the first ring by a spring and comprising a distal friction face,
a friction plate, connected to the free proximal free of the connected driving parts,
so that the setting in rotation of the driving part only occurs when the friction plate is in contact with the distal friction face, in particular on account of the application of the discharge head to the point at which the fastener is to be implanted.

19. The device according to claim 18, wherein it comprises a blocking finger and in that the second ring comprises longitudinal grooves forming safety catches, being configured to allow passing of the finger from one groove to the other on rotation of the actuating part at an angle A when the handle is actuated, and to act as abutment when the actuating part rotates in the reverse direction.

20. The device according to claim 16, wherein the assembly formed of the tubular body, the storage part, the actuating part and the discharge head form a fastener charger assembly which is independent of the shell and can be secured thereto by press-fitting the actuating part in the backstop system, in particular by press-fitting the proximal end of the actuating part in a hollow cylindrical end-piece secured to the friction plate.
